(19) United States
(12) Patent Application Publication
(10) Pub. No.: US 2022/0143332 A1
(43) Pub. Date: May 12, 2022

(54) SUPPLEMENT PLUS HYDROGEN INHALATION DEVICE

(71) Applicant: Aqua Bank CO.,LTD., Osaka-shi, Osaka (JP)

(72) Inventor: Takashi TAKEHARA, Osaka-shi, Osaka (JP)

(21) Appl. No.: 17/440,451

(22) PCT Filed: Mar. 19, 2020
(86) PCT No.: PCT/JP2020/012518

§ 371 Date: Sep. 17, 2021

(30) Foreign Application Priority Data
Mar. 19, 2019 (JP) 2019-051937

(51) Int. Cl. A61M 15/00; A61K 33/00; C25B 1/04; C25B 15/02; C25B 9/65; A61K 9/00

(52) U.S. Cl. CPC A61M 15/0003; A61K 33/00; A61K 9/0073; C25B 15/02; C25B 9/65; C25B 1/04

(57) ABSTRACT

A supplement inhalation and hydrogen inhalation device for enabling supplement inhalation administration and hydrogen inhalation includes: an electrolysis tank in which a pair of positive/negative electrodes are inserted, electrolysis tank being capable of storing an electrolytic solution or water, the pair of positive/negative electrodes being either energized or not energized; a supplement generation cartridge configured to be energized to be heated for releasing a supplement-containing gas; control means configured to control the pair of electrodes and/or the supplement generation cartridge each to be either energized or not energized; and an inhalation vessel configured to mix hydrogen and a supplement-containing gas and guide the gases to an oral inhalation opening or a nasal inhalation opening.

### TECHNICAL FIELD

The present invention relates to a supplement inhalation and hydrogen inhalation device enabling supplement inhalation administration and hydrogen inhalation.

### BACKGROUND ART

Recently, effect of hydrogen on removing active oxygens has attracted attention. In particular, some reactive oxygen species such as hydroxyl radicals are extremely toxic, and there is no removal system for them in the living body, and it has been known that hydrogen molecules reduce oxidative stress by selectively reducing these highly malignant reactive oxygen species in the living body, and may be utilized for prevention and treatment of various diseases.

Conventional methods of hydrogen intake into the body generally include intravenous dosage, oral administration of an aqueous solution, and gas inhalation. Recently, a portable electrolysis type hydrogen inhalation device (Patent Literature 1) has been attracting attention because it has a great intake amount of hydrogen, it is easy to control the intake amount depending on an energization time, and it is easy to use it regularly. In addition, the effects of the electrolytic hydrogen inhalation that are known include an effect of suppressing ischemic reperfusion injury (Non Patent Literature 1) and an effect of suppressing the progression of dementia (Patent Literature 2).

On the other hand, hydrogen inhalation is beneficial for maintaining health, and preventing and treating various diseases, but the effect is not immediate and many of the effects are not noticeable. It is also pointed out that it is difficult for users to maintain their inhalation habits on a daily basis because hydrogen is tasteless and odorless. Under these circumstances, a portable vapor and hydrogen inhalation device having a function of a vaping device, which is highly habitual to users, is also provided for enabling intake of supplements and hydrogen.

### CITATION LIST

### Patent Literature

Patent Literature 1: International Publication No. WO 2018/047889 A1
Patent Literature 2: International Publication No. WO 2018/151107 A1

### Non Patent Literature

Non Patent Literature 1
Noda et al., Protective effect of molecular hydrogen against white matter ischemic injury, Cerebral Blood Flow and Metabolism (Japanese journal of cerebral blood flow and metabolism) Vol. 26 No. 2, pp. 77-81

### SUMMARY OF INVENTION

### Technical Problem

However, there is also pointed out an aspect that even if a hydrogen inhalation device for purposes of maintaining health, and preventing and treating various diseases, is equipped with a function as an electronic cigarette that is harmful to health, it would not be possible to expect that non-smokers maintain a hydrogen inhalation habit, and it would be possible to damage the image of a health promotion device to hinder promotion of regular use as well as promotion of sales.

In view of these circumstances, an object of the present invention is to provide a supplement inhalation and hydrogen inhalation device enabling supplement inhalation administration, beneficial for health promotion, at the same time as hydrogen inhalation, and capable of increasing the body intake amount of hydrogen up to an appropriate amount by dissolving the supplement directly into the blood from the alveoli while promoting the maintenance of hydrogen inhalation habit.

### Solution to Problem

The present invention made to solve the aforementioned problems is a supplement inhalation and hydrogen inhalation device for enabling supplement inhalation administration and hydrogen inhalation, the device including:

an electrolysis tank in which a pair of positive/negative electrodes are inserted, the electrolysis tank being capable of storing an electrolytic solution or water, the pair of positive/negative electrodes being either energized or not energized;

a supplement generation cartridge configured to be energized to be heated for releasing a supplement-containing gas;

control means configured to control the pair of electrodes and/or the supplement generation cartridge each to be either energized or not energized; and

an inhalation vessel configured to mix hydrogen and a supplement-containing gas and guide the gases to an oral inhalation opening or a nasal inhalation opening, the hydrogen being generated by electrolyzing an electrolytic solution or water in the electrolysis tank when the pair of positive/negative electrodes are energized, the supplement-containing gas being released when the supplement generation cartridge is energized;

The supplement inhalation and hydrogen inhalation device of the present invention allows hydrogen inhalation and supplement inhalation administration into the body to be performed simultaneously or to be switched. In addition, since the power to be used for the electrolytic hydrogen generating device can also be used as the heating power for the supplement generation cartridge, the combination is favorable, and intake of supplements can be performed at the same time as hydrogen inhalation administration. Therefore, it is easy for users to maintain the habit of daily use of hydrogen inhalation. Furthermore, since the supplement can be dissolved directly into the blood from the alveoli, both the absorption rate and the absorption amount are increased as compared with oral ingestion. In particular, hydrogen inhalation has the advantage in which the blood vessels near the alveoli are dilated and blood flow is increased so that the supplement is easily absorbed. Furthermore, the medicinal ingredient that has been difficult to be absorbed due to gastric acid in the case of oral ingestion can be dissolved in the blood via the alveoli, which is also advantageous in that the immediate effect is increased.

In a specific example of the supplement inhalation and hydrogen inhalation device, the supplement generation cartridge is replaceable, has a tubular shape having a release port of supplement-containing gas at an upper part, and has a cartridge receiving portion, the cartridge receiving portion having a terminal at a bottom part, the terminal being capable of being electrically connected to a terminal at a lower part of the supplement generation cartridge when the supplement generation cartridge is inserted from above;

the electrolysis tank has the pair of positive/negative electrodes inserted into an inside from a lower part, and has, at an upper part, an injection port for an electrolytic solution or water, and a hydrogen generation port fluidically connecting to an air layer in the electrolysis tank; and

the inhalation vessel covers the hydrogen generation port of the electrolysis tank and the release port of the supplement generation cartridge, and forms an integral lid member such that the integral lid member mixes hydrogen and supplement-containing gas with environmental air and simultaneously guides the gases to the inhalation opening when the inhalation opening is inhaled, the hydrogen being released from the hydrogen generation port, the supplement-containing gas being released from the release port.

Furthermore, a particle diameter of a supplement in a supplement-containing gas generated when the supplement generation cartridge is heated is preferably 1 μm to 6 μm.

In the supplement inhalation and hydrogen inhalation device, it is necessary for the generated supplement to reach the bronchi or lungs from the mouth and nose without depositing in the trachea or respiratory tract. At this time, the particle diameter of the supplement is an important factor, and it is preferable that the particles have an aerodynamic particle diameter of about 6 μm. In addition, supplement particles need to reach the alveoli, to be efficiently absorbed through the lung mucosa, and to translocate into the blood. Here, the finer the particles, the worse the fluidity of the powder, so that there are concerns about deterioration of filling accuracy and handling ability during production. In that sense, it is necessary to have a supplement having a particle diameter of about 1 μm. Therefore, the particle diameter of the supplement in the supplement-containing gas generated by the supplement inhalation and hydrogen inhalation device is preferably 1 μm to 6 μm.

More preferably, the particle diameter of a supplement in a supplement-containing gas generated when the supplement generation cartridge is heated is less than 2.5 μm, a filter formed of a fibrous material is disposed at an outflow port of the supplement generation cartridge, the filter being capable of blocking fine particulate matter having a particle diameter of 2.5 μm or more.

As described above, supplements need to reach the bronchi or lungs from the mouth and nose not depositing in the trachea or respiratory tract, while it is difficult or costly to accurately generate such fine particles as fine particles having a particle diameter within a predetermined range. Furthermore, the fine particles have the property of aggregating, and as the particle diameter of the supplement increases, they do not reach the bronchi or lungs. Even if some reach there, there is also a problem in which they adhere to the alveoli and does not dissolve in the blood. On the other hand, recently, the problems of asthma, bronchitis, and chronic obstructive pulmonary disease caused by suspended solids in the atmosphere (so-called PM2.5) having a particle diameter of less than 2.5 μm have been attracting attention, and various filters that block this have been developed and are widely used. In the supplement inhalation and hydrogen inhalation device, from the viewpoint of difficulty in particle diameter generation of supplements and cost reduction, a filter for so-called PM2.5 countermeasures, which is widely used and utilized at low cost, is disposed at the outflow port of the supplement generation cartridge, so that only supplements with favorable particle diameters can reach the bronchi or lungs.

### Advantageous Effects of Invention

The supplement inhalation and hydrogen inhalation device of the present invention allows hydrogen inhalation and supplement inhalation administration into the body to be performed simultaneously or to be switched, This enables users to perform intake of supplements at the same time as hydrogen inhalation administration so that the users can easily maintain the habit of daily use of hydrogen inhalation. Furthermore, the supplement can dissolve directly into the blood from the alveoli so that both absorption rate and absorption amount increase as compared with oral ingestion. This enables intake of the medicinal ingredient, which has been difficult to be absorbed due to gastric acid in the case of oral ingestion, into the body via the alveoli as well, thus advantageously increasing the immediate effect.

### BRIEF DESCRIPTION OF DRAWINGS

FIG. 1 is a block diagram schematically illustrating an embodiment of a supplement inhalation and hydrogen inhalation device of the present invention.

FIGS. 2A-2D are a front view, left and right side views, and a plan view illustrating an embodiment of the supplement inhalation and hydrogen inhalation device of the present invention.

FIGS. 3A-3C are a schematic exploded view of a general-purpose vaping device including the supplement generation cartridge.

FIG. 4 is a schematic diagram illustrating a state of electrolysis in an electrolysis tank of the supplement inhalation and hydrogen inhalation device of the present invention.

FIG. 5 schematically illustrates a specific configuration of an electrode.

FIGS. 6A-6C are external photographic views of an example of the embodiment of the supplement inhalation and hydrogen inhalation device of the present invention.

### DESCRIPTION OF EMBODIMENTS

First, an example of a supplement inhalation and hydrogen inhalation device of the present invention is to be described below.

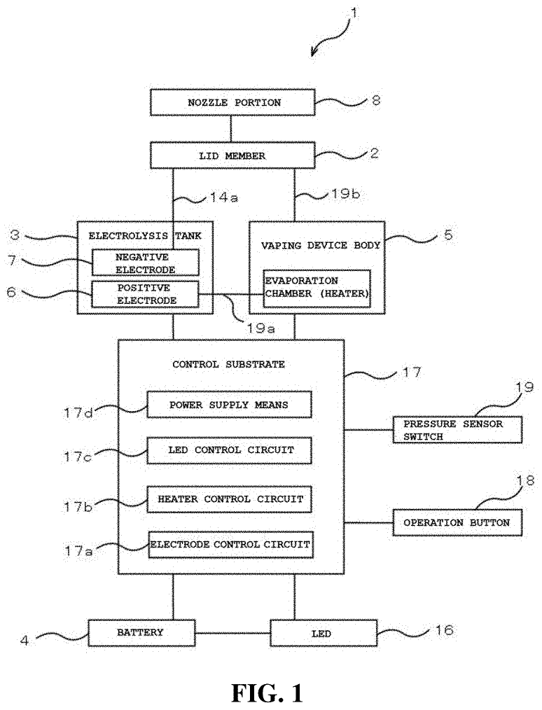

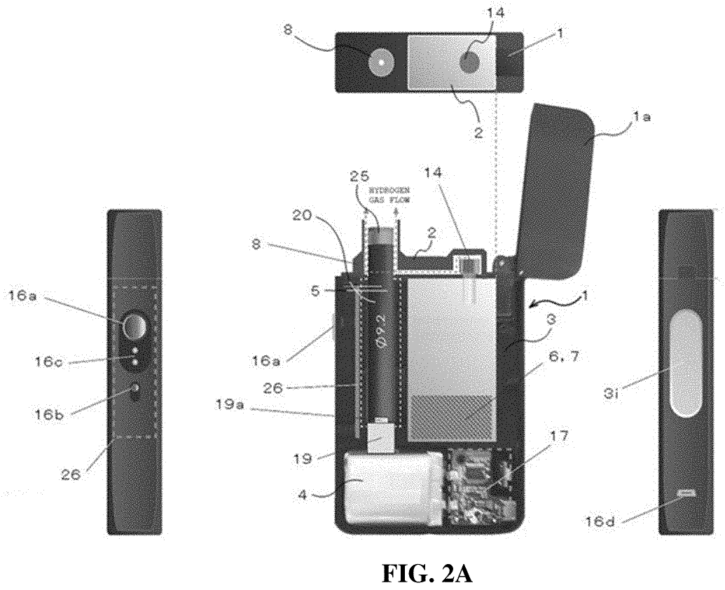

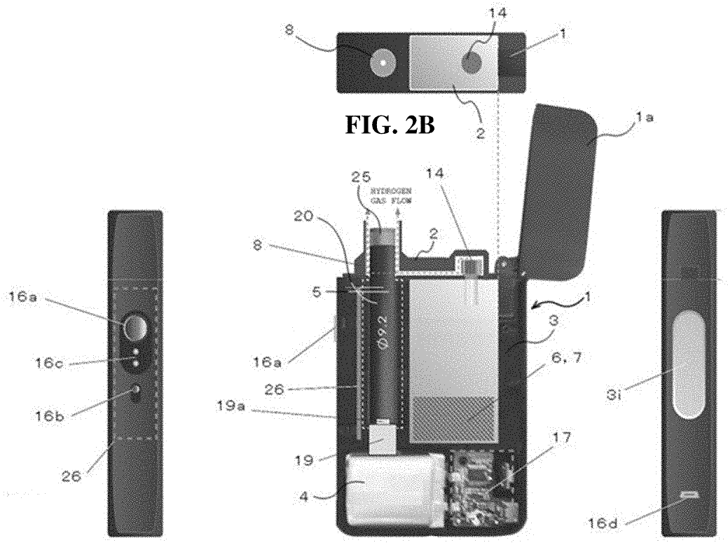

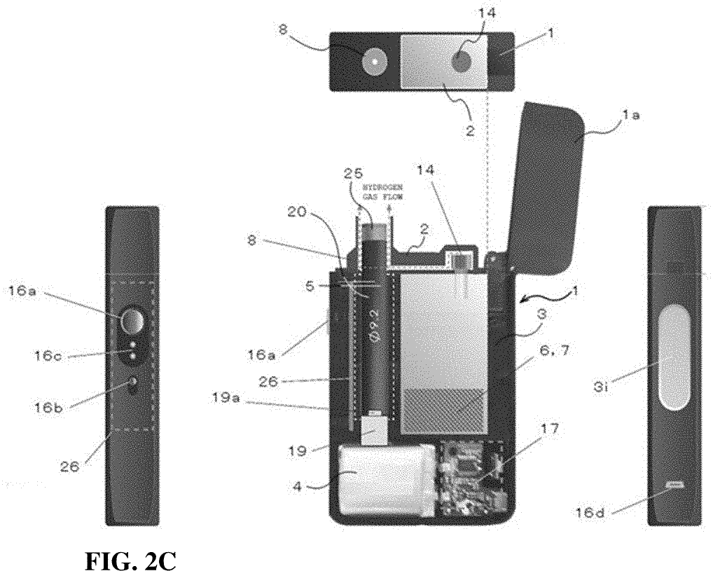

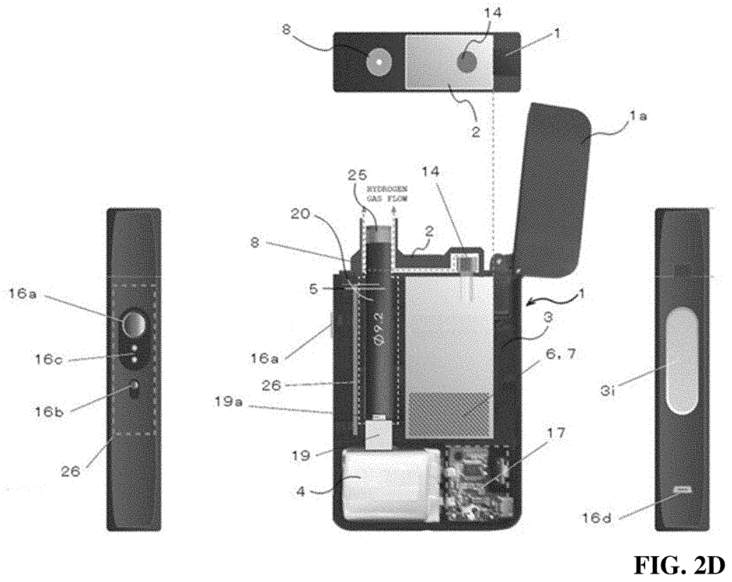

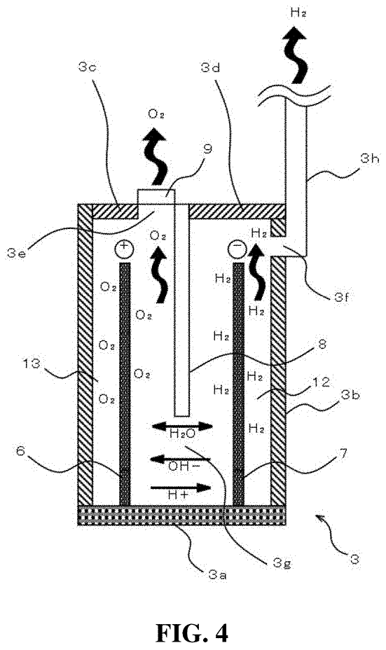

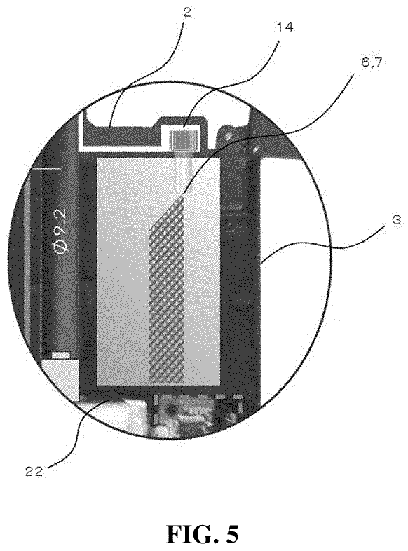

Embodiments of the supplement inhalation and hydrogen inhalation device of the present invention is to be illustrated and described below. FIG. 1 illustrates a block diagram schematically illustrating the embodiment, FIGS. 2A-2D illustrate six views of a typical example of the embodiment of FIG. 1, and FIG. 4 illustrates a schematic diagram illustrating a state of electrolysis in an electrolysis tank of the supplement inhalation and hydrogen inhalation device. FIG. 5 schematically illustrates a specific configuration of the electrode. Note that it is needless to say that the supplement inhalation and hydrogen inhalation device of the present invention is not limited to the device illustrated in the drawing, and also includes a device in which anything mentioned in the illustration and description is modified within the range of common sense.

As shown in FIG. 1, the supplement inhalation and hydrogen inhalation device includes a battery 4, an LED 16, control means 17, an electrolysis tank 3, a supplement generation cartridge 5, a mixing portion 2, and a nozzle portion 8. The battery 4 is of a charging type, and a pair of positive/negative electrodes 6 and 7 are disposed in the electrolysis tank 3. The positive/negative electrodes 6 and 7 are supplied with power from the battery 4 via the control means 33, and the LED 16 is connected to the battery 4. The control means 17 includes an electrode control circuit 17a, a heater control circuit 17b, an LED control circuit 17c, and power supply means (a power supply circuit) 17d.

A pressure sensor switch 19 is provided at the bottom part of the receiving portion of the supplement generation cartridge 5, and when the lower end of the supplement generation cartridge presses the pressure sensor switch 19, the power of the battery 4 is supplied to the supplement generation cartridge 5 by the power supply means 17d of the control substrate 17.

When a user operates the operation button 18, the electrode control circuit 17d accordingly controls energization/non-energization of a pair of electrodes 6 and 7 in the electrolysis tank 3, and the electric power supply means 17d varies the amount of power supplied from the battery 4 to supply power to the electrodes 6 and 7. When power is supplied to the pair of electrodes 6 and 7, the water stored in the electrolysis tank 10 is electrolyzed, so that oxygen is generated on the positive electrode 6 side, and hydrogen is generated on the negative electrode 7 side.

Hydrogen generated from the negative electrode 7 flows into the lid member 2 via the attachment 14 on the upper part of the electrolysis tank 3. The oxygen generated from the positive electrode 6 is vented.

In the supplement generation cartridge 5, when the pressure sensor switch 19 is turned on, the heater inside the supplement generation cartridge 5 is supplied with power from the battery 4 by the power supply means 17d, and the heater heats a cartridge, attached to an internal vapor chamber (not shown), that has adsorbed a supplement. When the cartridge on which the supplement is adsorbed is heated by the heater, the supplement-containing vapor is generated. Note that, in the present embodiment, the cartridge 25 on which the supplement is adsorbed and the supplement generation cartridge 5 are illustrated as separate parts, but there is also adopted an example in which a cartridge having both integrated therein is formed, and the supplement-containing vapor is released as the water inside evaporates due to heating.

The supplement-containing vapor generated by the supplement generation cartridge 5 is released into the mouth by inhaling the nozzle portion 8. At this time, due to a negative pressure generated by inhalation, hydrogen released from the attachment 4 flows in the lid member 2, and passes through the gap between the periphery of the upper part of the supplement generation cartridge 5 exposed in the lid member 2 and the inner wall of the nozzle portion 8, mixes with the supplement-containing air, and is guided into the mouth. It is also conceivable to heat this to generate supplement-containing vapor (which may be aromatic).

FIGS. 2A-2D show a specific configuration example of the supplement inhalation and hydrogen inhalation device 1 with the supplement generation cartridge 5 inserted therein. FIG. 2A illustrates a front view, FIG. 2B a top view, FIG. 2C a left side view, and FIG. 2D a right side view of the supplement inhalation and hydrogen inhalation device 1. FIG. 2A is a state in which the lid member 2 of the supplement inhalation and hydrogen inhalation device 1 is removed, and there is a tubular cartridge receiving portion (hereinafter, also referred to as “receiving portion”) 20 extending downward from an opening on the upper right side with the lid member 2 removed (opened). The supplement generation cartridge 5 is inserted into the receiving portion 20. The supplement generation cartridge 5 is a body portion of a general-purpose tubular heating type electronic cigarette. A general-purpose heating type electronic cigarette is to be illustrated below.

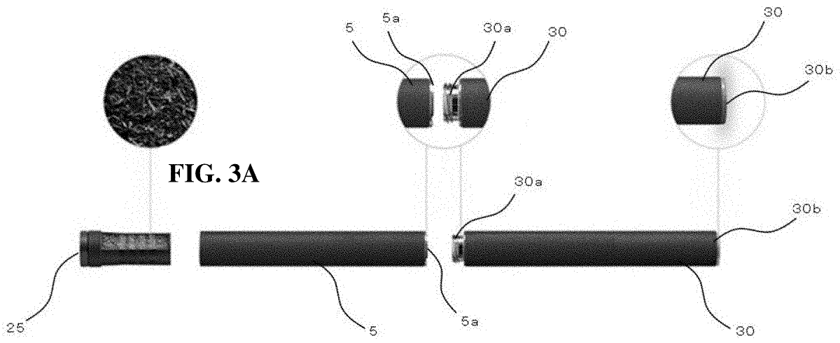

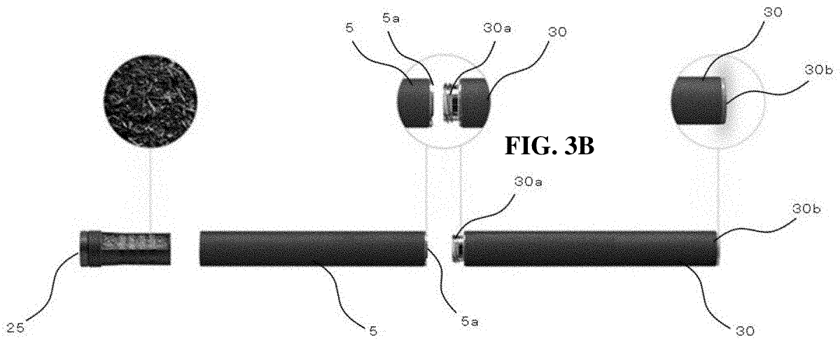

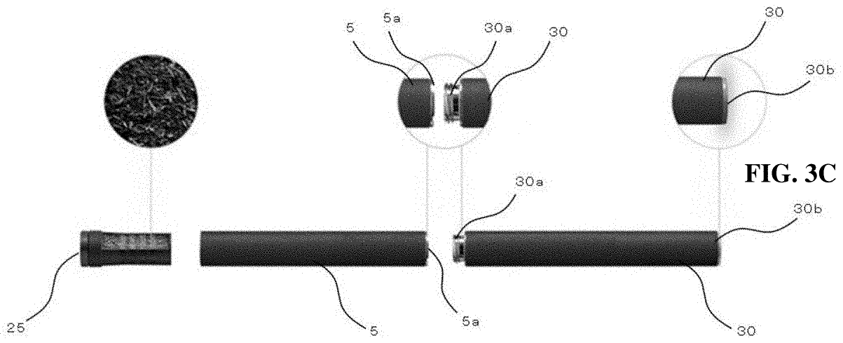

The supplement generation cartridge 5 can also be used alone for supplement inhalation administration by replacing a vaping body portion of the heating type electronic cigarette 100 of FIGS. 3A-3C with the supplement generation cartridge 5. As shown in FIGS. 3A-3C, the supplement generation cartridge 5 includes three members, which are a cartridge for aroma 25, the supplement generation cartridge 5, and a battery portion 30, illustrated from the left, and these members are connected in the longitudinal direction. The cartridge for aroma 25 is a disposable cylindrical cartridge in which an aromatic in the form of granules and so on is enclosed, and is provided with a hole for releasing vapor.

At the time of supplement inhalation administration, the cartridge for aroma 25 is pushed from the upper end (left end in FIGS. 3A-3C) of the supplement generation cartridge 5, and inserted into an internal hollow vapor chamber (not shown). The inner wall of the upper part of the substantially cylindrical supplement generation cartridge 5 is formed with a tapered slope that opens upward so that the cartridge for aroma 25 can be easily inserted, or the bottom part of the cartridge for aroma 25 is provided with an insertion opening corresponding to the cavity surface of the vapor chamber so that it can be easily fixed to the chamber and the contact area is expanded to facilitate heat conduction. When the supplement generation cartridge 5 is inhaled in the upper part and a negative pressure is generated, it is turned on, so that power is supplied from the rechargeable battery in the battery 30, which will be described later, and the vapor chamber is heated by the heater. Then, the moisture in the cartridge for aroma 25 is vaporized and aromatic components are released. In that sense, the supplement generation cartridge 5 and the cartridge for aroma 25 are sufficient as a device for oral intake of supplement-containing vapor by inhalation. The supplement generation cartridge 5 is a semi-disposable device in which the cartridge for aroma 25 can be used five times in the example of FIGS. 3A-3C.

The lower end (right end in FIGS. 3A-3C) of the supplement generation cartridge 5 can be connected to the battery 30. The battery 30 and the supplement generation cartridge 5 have a shape of one cylindrical rod in a state in which they connect together. As shown in the photographic example of FIG. 3B, an attachment 5a is provided at the lower end of the supplement generation cartridge 5, and an attachment 30b is provided at the upper end of the battery 30, so that they can be connected to each other. In the example of FIGS. 3A-3C, a recess part provided with a screw groove is formed in the attachment 5a on the supplement generation cartridge 5 side, a projection part provided with screw threads is formed on the attachment 30a on the battery 30 side, and coaxial rotations insert the battery 30 into the supplement generation cartridge 5 to connect them.

In addition, the attachment 5a also has a function as a terminal electrically connected to the heater in the supplement generation cartridge 5, and the attachment 30a also has a function as a terminal electrically connected to the rechargeable battery in the battery 30. Therefore, when the attachments 5a and 30a are connected, power is supplied from the rechargeable battery in the battery 30 to the heater in the supplement generation cartridge 5, and the cartridge for aroma 25 is heated via the vapor chamber. When a negative pressure generated by the inhaling of the upper end is applied to the supplement generation cartridge 5, power is supplied to the internal heater from the battery 30, and at the same time, an LED 30b at the lower end of the battery 30 lights up while power is supplied from the battery 30.

Here, returning to FIGS. 2A-2D, the supplement inhalation and hydrogen inhalation device 1 is to be described. The supplement inhalation and hydrogen inhalation device 1 has the supplement generation cartridge 5 of general-purpose heating type electronic cigarette of FIGS. 3A-3C described above, inserted into the receiving portion 20. A pressure sensor switch 19 is disposed at the bottom part of the receiving portion 20, and a convex screw 19a having the same shape as the attachment 30a is provided at the upper end of the pressure sensor switch 19 as an electric terminal. When the pressure sensor switch 19 is pressed, power is supplied to the convex screw 19a from the rechargeable battery (lithium battery) 4. In that sense, the rechargeable battery 4 functions as a substitute for the battery 30 illustrated in FIGS. 3A-3C. In other word, when the supplement generation cartridge 5 is inserted into the receiving portion 20 and the supplement generation cartridge 5 is pressed from above with the attachment 5a fastened to the convex screw 19a, power of the rechargeable battery 4 is supplied to the supplement generation cartridge 5 via the convex screw 19a, to heat the cartridge for aroma 25 so that the supplement-containing vapor can be inhaled.

The right side part of the supplement inhalation and hydrogen inhalation device 1 (see FIG. 2C) is provided with an electronic cigarette on/off switch 16c, an LED indicator 16b, and a main power source/hydrogen button 16a. The electronic cigarette on/off switch 16c is an on/off switch of the pressure sensor switch 19, so that when it is turned on, the power of the rechargeable battery 4 would be supplied to the supplement generation cartridge 5 if the attachment 5a at the lower end of the supplement generation cartridge 5 is pressed with the convex screw 19 connected thereto, but, when it is turned off, the power would not be supplied from the rechargeable battery 4 even if the pressure sensor switch 19 is pressed. The main power source/hydrogen button 16a is a button-type power supply switch of the positive/negative poles 6 and 7 in the electrolysis tank 3, which is to be described below, and the main power source, and is used as both on/off of the main power source and on/off of power supply to the positive/negative poles 6 and 7 depending on the pressing manner/time.

In this example, when the main power source/hydrogen button 16a is held down for 3 seconds, the positive/negative poles 6 and 7 are energized for 5 minutes to generate hydrogen, and when it is pressed three times for two seconds, the main power source is turned off. The main power source is automatically turned off after 20 minutes if the operation of turning it off is not performed. Furthermore, the main power source/hydrogen button 16a lights up while hydrogen is generated, and has a function of displaying the remaining amount of the rechargeable battery 4 according to the lighting color. In this example, when the remaining battery level is 20 to 80%, it lights up in blue, and when the remaining battery level is 80 to 100%, it lights up in white. In addition, the LED indicator 16b is provided with two LEDs each at upper and lower sides, the upper side LED lights up when power is supplied to the positive/negative poles 6 and 7 in the electrolysis tank 3, and the lower side LED lights up when the pressure sensor switch 19 is turned on and the supplement generation cartridge 5 is energized. The lighting of the electronic cigarette on/off switch 16c, the LED indicator 16b, and the main power source/hydrogen button 16 is controlled by the internal indicator substrate 26.

As described above, when the pressure sensor switch 19 is turned on, the power from the rechargeable battery 4 is also supplied to the pair of positive/negative electrodes 6 and 7 by the control substrate 17. As shown in FIG. 2C, the pair of positive/negative electrodes 6 and 7 may be disposed horizontally on the inner bottom part of the electrolysis tank 3, or may be disposed vertically as shown in FIG. 5. The rechargeable battery 4 can receive power from the USB terminal 16d on the side part of the supplement inhalation and hydrogen inhalation device 1, and is charged (see FIG. 2D).

Next, with reference to FIG. 4, description is to be made below on the configuration inside the electrolysis tank 3 and the state of electrolysis in the electrolysis tank 3 when the positive/negative electrodes 6 and 7 are energized. As shown in FIG. 4, the electrolysis tank 3 in which water is stored includes: a tubular member 3b that is hollow and longitudinally extends; a bottom member 3a that closes the bottom part of the tubular member 3b; and the lid members 3c and 3d that close the upper part of the tubular member 3a (3c and 3d may be integrally formed). When the positive/negative electrodes 6 and 7 are energized, oxygen (O₂) is generated in the vicinity of the positive electrode 6 and hydrogen (H₂) is generated in the vicinity of the negative electrode 7. Since the generated oxygen and hydrogen have a lighter specific gravity than water, they move upward and each moves to a gap of 3g. Here, the electrolysis tank 3 is provided with a partition member 8 extending downward from the upper end thereof and dividing the electrolysis tank 3 into a hydrogen gas generating layer 12 on the negative electrode 7 side and an oxygen gas generating layer 13 on the positive electrode 6 side. The lower end of the partition member 8 is provided with a gap 3g from the upper surface of the bottom member 3a so as to fluidically connect the hydrogen gas generating layer 12 and the oxygen gas generating layer 13.

The partition member 8 prevents the mixing of oxygen and hydrogen in the electrolysis tank 3 during the upward movement of oxygen and hydrogen. On the other hand, in the lower part of the aforementioned gap 3g, which is not partitioned by the partition member 8, provided below the partition plate 8, water (H₂O) can freely move, that is, ions (“OH⁻” and “H⁺”) can move, which is required for generation of oxygen and hydrogen. In this way, the partition member 50 achieves prevention of mixing of oxygen and hydrogen while electrolysis is performed.

The lid member 3c closes the upper part of the oxygen gas generating layer 13, but is provided with an opening 3e between a part of the lid member 3c or the lid member 3c and the partition member 8 or the tubular member 3b. The opening 3e is closed by the oxygen permeable membrane 9. Therefore, if hydrogen leaks from the hydrogen gas generating layer 12 to the oxygen gas generating layer 13 due to the gap 3g or the like, the gas released to the outside by the oxygen permeable membrane 9 would be limited to oxygen. The oxygen permeable membrane 9 may be disposed at the electrolytic solution injection port/hydrogen generation port 14 (to be described below) shown in FIGS. 2A-2D, but is preferably disposed in a hole dedicatedly provided in the electrolysis tank 3.

In addition, also in the hydrogen gas generating layer 12, the upper part of the hydrogen gas generating layer 12 is closed by the lid member 3d, but an opening 3f is provided in the upper part of the tubular member 3b on the hydrogen gas generating layer 12 side. The opening 3f is connected to the bypass channel 3h. Therefore, the hydrogen in the hydrogen gas generating layer 12 generated at the negative electrode 7 flows into the bypass channel 3h and flows upward.

Regarding the hydrogen channel from the opening 3f to the bypass channel 3h in FIG. 4, in the example of FIGS. 2A-2D, the electrolytic solution injection port/hydrogen generation port 14 corresponds to the opening 3f, and the gap between the upper part of the electrolysis tank 3 and the lid member 2 corresponds to the bypass channel 3h. As described above, the electrolytic solution injection port/hydrogen generation port 14 has a function as an inlet for injecting the electrolytic solution or water into the electrolysis tank 3 and a function as an opening 3f for releasing hydrogen in the electrolysis tank 3 to the outside. The electrolytic solution injection port/hydrogen generation port 14 has a shape in which screws can be detachably fastened, and the electrolytic solution or water is injected through the opening 3f in a state where the screws are loosened and removed. In addition, leakage of the electrolytic solution and the like in the electrolysis tank 3 is prevented with the screws being fastened, but hydrogen is released from a permeable membrane for gas such as hydrogen (not shown) that closes a hole or the like separately provided in the electrolytic solution injection port/hydrogen generation port 14.

The released hydrogen flows in the lid member 2 in the left direction (supplement generation cartridge 5 direction) as shown by the dotted line in FIGS. 2A-2D. The lid member 2 has a tubular nozzle portion 2a having an opening at the upper end and projecting at the left end, and is a removable integral member disposed on the upper part of the electrolysis tank 3, covering the upper end of the supplement generation cartridge 5 and the electrolytic solution injection port/hydrogen generation port 14. When the lid member 2 is mounted on the upper part of the electrolysis tank 3, the nozzle portion 2a is in a state in which the upper end of the supplement generation cartridge 5 is nested in the opening of the nozzle portion 2a with a gap 26 around it. When the nozzle portion 2a is inhaled, hydrogen (high-concentration hydrogen-containing air) flowing from the electrolytic solution injection port/hydrogen generation port 14 rises in the gap 26 and is released to the outside (see the dotted line in FIGS. 2A-2D). This enables intake of high-concentration hydrogen. In addition, when power is supplied to the supplement generation cartridge 5, this enables intake of a gas mixture of high-concentration hydrogen, and a supplement and/or aromatic containing air from the cartridge for aroma 25. The supplement inhalation and hydrogen inhalation device 1 includes a cover 1a that can be opened and closed, and the example of FIGS. 2A-2D illustrate a state in which the cover 1a is opened. An opening (electrolytic solution check window) 3i for observing the liquid amount in the electrolysis tank 3 is provided on a side part of the supplement inhalation and hydrogen inhalation device 1 so that the amount of liquid in the electrolysis tank 3 can be visually recognized.

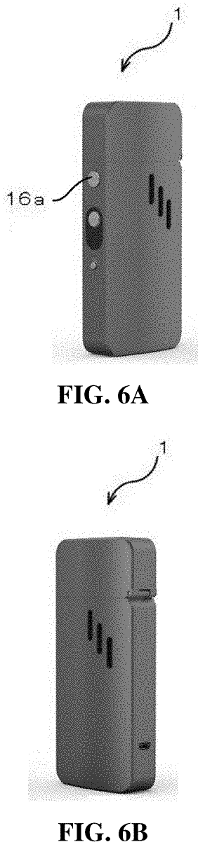

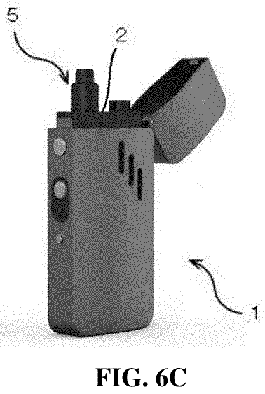

For reference, FIGS. 6A-6C show external photographic views of an example of the embodiment of the supplement inhalation and hydrogen inhalation device 1 of the present invention. FIG. 6A illustrates the supplement inhalation and hydrogen inhalation device 1 viewed from diagonally left front, FIG. 6B illustrates the supplement inhalation and hydrogen inhalation device 1 viewed from diagonally right front, and FIG. 6C illustrates a state in which the cover 1a of the supplement inhalation and hydrogen inhalation device 1 is opened, viewed from diagonally left front of (a). Although there are some parts different from the example of FIGS. 2A-2D in terms of design, the main structure is substantially the same, and the reference numerals given to FIGS. 6A-6C are the same as those of the example of FIGS. 2A-2D.

As described above, the embodiment of the supplement inhalation and hydrogen inhalation device of the present invention is illustrated and described, but the present invention is not limited to this, and those skilled in the art would understand that other modifications and improvements can be obtained without departing from the spirit and teachings described in the claims and the specification.

### REFERENCE SIGNS LIST

1 supplement inhalation and hydrogen inhalation device
2 lid member
3 electrolysis tank
4 rechargeable battery
5 supplement generation cartridge
6 positive electrode
7 negative electrode
8 nozzle portion
17 control means
18 operation button (operation means)
19 pressure sensor switch
19a convex screw
20 cartridge receiving portion
25 cartridge with supplement adsorbed

1. A supplement inhalation and hydrogen inhalation device for enabling supplement inhalation administration and hydrogen inhalation, the device comprising:
an electrolysis tank in which a pair of positive/negative electrodes are inserted, the electrolysis tank being capable of storing an electrolytic solution or water, the pair of positive/negative electrodes being either energized or not energized;
a supplement generation cartridge configured to be energized to be heated for releasing a supplement-containing gas;
control means configured to control the pair of electrodes and/or the supplement generation cartridge each to be either energized or not energized; and
an inhalation vessel configured to mix hydrogen and a supplement-containing gas and guide the gases to an oral inhalation opening or a nasal inhalation opening, the hydrogen being generated by electrolyzing an electrolytic solution or water in the electrolysis tank when the pair of positive/negative electrodes are energized, the supplement-containing gas being released when the supplement generation cartridge is energized;

2. The supplement inhalation and hydrogen inhalation device according to claim 1, wherein:
the supplement generation cartridge is replaceable, has a tubular shape having a release port of supplement-containing gas at an upper part, and has a cartridge receiving portion, the cartridge receiving portion having a terminal at a bottom part, the terminal being capable of being electrically connected to a terminal at a lower part of the supplement generation cartridge when the supplement generation cartridge is inserted from above;
the electrolysis tank has the pair of positive/negative electrodes inserted into an inside from a lower part, and has, at an upper part, an injection port for an electrolytic solution or water, and a hydrogen generation port fluidically connecting to an air layer in the electrolysis tank; and
the inhalation vessel covers the hydrogen generation port of the electrolysis tank and the release port of the supplement generation cartridge, and forms an integral lid member such that the integral lid member mixes hydrogen and supplement-containing gas with environmental air and simultaneously guides the gases to the inhalation opening when the inhalation opening is inhaled, the hydrogen being released from the hydrogen generation port, the supplement-containing gas being released from the release port.

3. The supplement inhalation and hydrogen inhalation device according to claim 1, wherein a particle diameter of a supplement in a supplement-containing gas generated when the supplement generation cartridge is heated is 1 μm to 6 μm.

4. The supplement inhalation and hydrogen inhalation device according to claim 1, wherein:
a particle diameter of a supplement in a supplement-containing gas generated when the supplement generation cartridge is heated is smaller than 2.5 μm; and
a filter formed of a fibrous material is disposed at an outflow port of the supplement generation cartridge, the filter being capable of blocking fine particulate matter having a particle diameter of 2.5 μm or more.
